(19) United States
(12) Patent Application Publication
(10) Pub. No.: US 2006/0122466 A1
(43) Pub. Date: Jun. 8, 2006

(54) Telemedicine system comprising a modular universal adapter

(76) Inventors: Trong-Nghia Nguyen-Dobinsky, Berlin (DE); Peter Hufnagl, Berlin (DE); Martin Schultz, Berlin (DE)

Correspondence Address: WOLF, BLOCK, SCHORR & SOLIS-COHEN LLP, 1650 ARCH STREET, 22ND FLOOR, PHILADELPHIA, PA, 19103-2334, US

(21) Appl. No.: 10/524,714

(22) PCT Filed: Aug. 12, 2003
(86) PCT No.: PCT/EP03/08961

§ 371 Date: Aug. 22, 2005

(30) Foreign Application Priority Data
Aug. 15, 2002 (DE) 102376921

(51) Int. Cl. A61B 5/00; A61B 5/02; A61B 5/04

(52) U.S. Cl. 600/300; 600/509; 600/323; 600/485; 600/365

(57) ABSTRACT

The present invention comprises a modular telemedicine system with a universal adapter that connects diagnostic, identification and audiovisual communication function modules, at least one of which is present at the patient's location, to a variable process module that performs data transmission, processing and output. The connection of the system, which can be used anywhere, to a physician's receiving center facilitates the mobile telemedical treatment of patients. Preferably, the system comprises a plurality of function modules for the recording of data. Simplified, basic operation of all function modules is made possible through a hard-wired connection between the function module and the universal adapter, and data can be transmitted to corresponding process modules wirelessly or hard-wired. Each function module can be used separately or in combination with the universal adapter and the process module. The function module and universal adapter have a central processor for data processing and non-mechanical storage elements for data storage as well as control and signaling elements on the outside of the module for operation.

The present invention comprises a modular telemedicine system with a universal adapter that connects diagnostic, identification and audiovisual communication function modules, at least one of which is present at the patient location, to a variable process module that performs data transmission, processing and output. Telemedical treatment of persons is made possible by the connection of the telemedicine system according to the invention to a receiving center located at the doctor's office.

The combination of a universal adapter with individual modules and process modules that can vary in number and function represents a system that can be implemented in a very flexible manner. In the process, the small physical tolerances of system components—universal adapter, function and process modules—facilitate the mobile application of the telemedicine system.

Reference is made in this invention to the older German patent application by this inventor entitled “Telemedicine System” (DE 101 54 908.3) for information concerning the configuration of the function modules.

This device is used in situations that require diagnostic testing using medical devices for physicians to make determinations on acutely ill persons, chronically ill patients and healthy patients, said testing having been impossible to do until now due to the situation or which is possible using the current art only at an unreasonably large degree of technical effort. Such situations include mobile medical treatment of individuals or groups of persons who are out of the country, direct treatment of chronically ill patients at their homes, as well as regular prophylactic self-examination by healthy individuals.

The special design of the universal adapter connection and the correspondingly uniform configuration of the function module connections make it possible to connect any function module to the universal adapter, and thus to the process modules, in exactly the same way. In the process, the universal adapter and function module connections are designed such that they can be intuitively connected correctly even by untrained persons, and that the plugged connection is robust.

Furthermore, the universal adapter is designed such that it allows basic, easy operation of any function module in a uniform manner. The universal adapter reduces the control of different diagnostic devices to a two-knob control system. For all function modules, data recording, transmission and status inquiries can be carried out using two control knobs located on the outside of the universal adapter. Operation is supported by corresponding visual signal elements (LEDs] and acoustic signals as well as a function display to indicate the operating state and to reference the operating steps.

This universal control system provided via the universal adapter provides inexperienced persons the ability to immediately handle a wide variety of function modules/diagnostic devices. Operation or configuration of the universal adapter and function modules can be expanded to include both remote access by the physician's receiving center as well as by corresponding process modules such as PCs or PDAs.

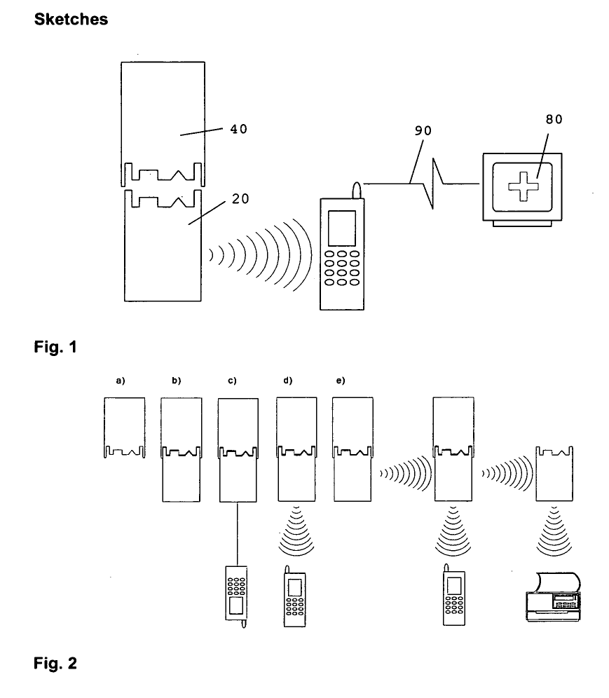

The function modules, at least one of which is present in the telemedicine system according to the invention, are generally hand-held, transportable medical measuring devices used to record diagnostic parameters such as electrophysiological cardiac cycle, blood pressure, body temperature or blood oxygen saturation. The modules are used either by the patients themselves or by another person present with the patient. Device-specific sensors record the measurements, which are then stored on non-mechanical storage media in the device. All function modules are fully functional individual devices and can be used independent of the remaining components of the system. To enable this, all function modules have corresponding control elements and signal elements as well as a variable display. Connecting the function module to a corresponding process module—preferably a mobile telephone—by means of the universal adapter allows the data to be transmitted to the physician's receiving center and makes it available to the physician for diagnostic and/or therapeutic decisions. The entire telemedicine system [function module, universal adapter, process module] is controlled in a simple manner using the universal adapter (FIG. 1

The following diagnostic function modules are possible candidates for integration into the system: an electrocardiograph, a pulsoximeter, a spirometer, a blood pressure measurement device, a thermometer, a cardiotocograph, a heart beat monitor [event recorder], a blood sugar measuring device as well as other devices. Other modules are integrated into the system according to connection design requirements.

In addition to diagnostic function modules, other types of function modules exist in the telemedicine system. This includes a module for personal identification, for example. It is possible, by means of the module, to authenticate the person doing the transmitting and/or to sign the transmitted data using biometric processes [such as fingerprinting, iris s can] and by reading identification cards. Expanding the telemedicine system to include such an identification module provides an advantage in comparison to identification mechanisms internal to the device in common use today—such as transmitting the phone number of the communication device or the device ID of the diagnostic function module—in that the telemedicine system can even be used by groups of persons, there being the ability to assign measured data to individual persons.

Other function module possibilities include the recording of picture, video and audio data. The transfer of these data to the physician's receiving center allows the physician to make diagnostic and therapeutic decisions.

The modular construction of the telemedicine system permits flexible usage in various scenarios. The telemedicine system is equipped with function modules according to need. In the process, the medical requirements determine whether the system is to contain only a particular diagnostic function module, to monitor blood sugar for instance, or if it is to include all available function modules and thus enable a wide spectrum of diagnostic tests and comfortable audiovisual communication.

Basically, many different mobile telecommunication devices as well as data processing and output devices [PCs, printers, etc.] can be used as process modules. Thus, a conventional GSM or GPRS mobile telephone can be used for data transmission, as can a UMTS or satellite telephone.

The exchange of data between the function modules and the universal adapter is done through a hard-wired connection. The corresponding I/O connections on the universal adapter and on the function modules are designed in accordance with a universal standard [such as the USB-2.0 standard].The transfer protocol used is a standard internet protocol [such as TCP/IP].

The connection of the universal adapter to the process modules can be hard-wired —using device-specific I/O connections—or wireless [Bluetooth, WLAN].

By integrating appropriate application software into the universal adapter, the system can generate and monitor diagnostic and therapeutic plans. This process must take into account the function modules that are registered, and thus available, in the system. The function modules are automatically registered on the universal adapter when they are interconnected.

There is a huge advantage to the system according to the invention when simultaneously using more than one telemedicine system in that the universal adapters can communicate with one another. This enables the common usage of individual process modules, for example. In this case, transmission of data from the universal adapters to the process module is done through a single common transmission path of one universal adapter. Thus, it is also possible to process or transmit numerous diagnostic data from different function modules at the same time using one process module (FIG. 2

Below, the individual components of the telemedicine system are described in detail with reference to the sketches.

FIG. 1: shows the basic design of the modular universal adapter—telemedicine system

FIG. 2: shows various application possibilities of the individual system components

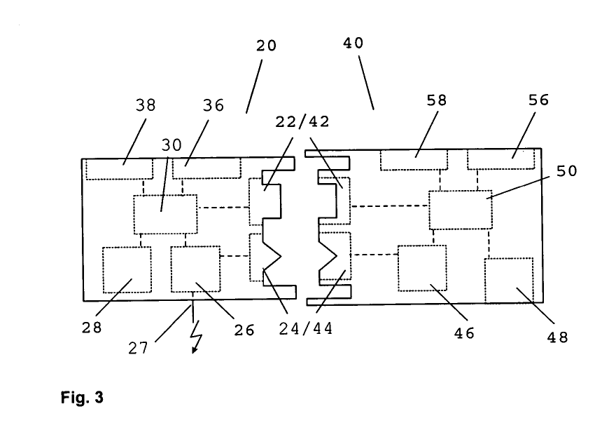

FIG. 3: shows a detailed schematic design of the universal adapter and the function module

The telemedicine system according to the invention 10 has a modular design. In essence, it comprises three components, a function module 40, a universal adapter 20, and a process module 60. The telemedicine system 10 can connect to a physician's receiving center 80 via a wireless transmission path 90 with the aid of corresponding process modules 60 (FIG. 1

The function module 40 is a module of varying function and number that represents either a medical diagnostic module that records specific diagnostic parameters, an identification module for personal identification, or an audiovisual communication module.

Medical diagnostic modules can include an electrocardiograph, a pulsoximeter, a spirometer, a blood pressure measurement device, a thermometer, a cardiotocograph, a heart beat monitor [event recorder], a blood sugar measuring device or other devices.

All function modules are identified through common technical features. Thus, all devices have identical connections 42/44 for the universal adapter 20. The bi-functional connection enables charging of the module's internal battery as well as hard-wired data transfer. Control elements 56 are located on the outside of the module to allow measurement operations. In addition, all function modules 40 have displays 58 that vary in shape and size to display measurements and/or to check function. The diagnostic function modules contain corresponding sensors 48 to measure the diagnostic parameters. If the function module is designed as an identification module, it has a reader that reads biometric identification features [fingerprint, iris] or identification cards.

Another type of function module 40 equipped with sensors to record acoustic and video signals and with a large color display and loudspeaker enables audiovisual communication.

The universal adapter 20 contains a universal I/O connection 22/24 [for example USB-2.0 standard] for the function modules 40, through which the data is transferred hard-wired and the battery of the function modules 40 is charged. Data is exchanged with the process module 60 via a wireless connection [for example Bluetooth, WLAN]. Use of the module is supported by control elements 36 and function displays 38 on the outside of the module. Furthermore, the universal adapter 20 has its own battery 26 so that it can be operated independent of network power. The batteries of the universal adapter 20 and function modules 40 are charged using a charging cable 27 connected to the universal adapter 20. The different application possibilities of the system derive from the modular design of the telemedicine system 10. Thus, it is possible to use the function modules 40 directly [FIG. 2a], or to connect all function modules 40 sequentially to the universal adapter 20 and to operate them by means of the universal adapter 20 in an identical manner [FIG. 2b]. Such operation includes mainly basic operating steps, such as data recording, data transmission and status inquiries. Furthermore, the use of the modular universal adapter—telemedicine system as such includes the wireless or hard-wired connection of the universal adapter 20 to a process module 60 [FIG. 2c, d]. This permits the connection to the physician's receiving center 80 over transmission path 90 to be accomplished using suitable process modules. Moreover, wireless communications of universal adapters 20 with one another permits common usage of individual process modules 60 that are present [FIG. 2e].

Parts List

10 Telemedicine system
20 Universal adapter
22 I/O connection (for example USB)
24 Power connection (for example USB)
26 Battery
27 Charging cable
28 Wireless interface (WLAN/Bluetooth)
30 Central processor
36 Control elements
38 Function display
40 Function module
42 I/O connection (for example USB)
44 Power connection (USB)
46 Battery
48 Measurement sensor
50 Central processor
56 Control elements
58 Display
60 Process module
80 Receiving center
90 Transmission path

1. A modular universal adapter telemedicine system comprising
at least one function module for at least one item selected from the group consisting of diagnostic testing, communication and identification;
a process module for providing at least one item selected from the group consisting of data output, data processing and data transmission; and
a universal adapter to connect the function modules to the process module.

2. A modular universal adapter according to claim 1, characterized in that the data collected during use of the function modules is at least one item selected from the group consisting of measurable medical parameters, identification features, audiovisual data and geographic position data.

3. A modular universal adapter—telemedicine system according to claim 1, characterized in that the at least one function module can be easily operated in the same manner using the universal adapter by means of a two-knob controller.

4. A modular universal adapter telemedicine system according to claim 1, characterized in that the at least one function module comprises at least one diagnostic function module for the purposes of medical diagnostic testing.

5. A modular universal adapter telemedicine system according to claim 4, characterized in that the diagnostic function module is an electrocardiograph, a pulsoximeter, a spirometer, a blood pressure measurement device, a thermometer, a cardiotocograph, a heart beat monitor or other event recorder, or a blood sugar measuring device.

6. A modular universal adapter telemedicine system according to claim 1, characterized in that the at least one function modules includes at least one identification module to record an identification features of a patient.

7. A modular universal adapter telemedicine system according to claim 6, characterized in that the identification module involves at least one function selected from the group consisting of recording biometric data of the patient and reading identification cards.

8. A modular universal adapter telemedicine system according to claim 1, characterized in that the at least one function modules includes at least one communication module for audiovisual communication.

9. A modular universal adapter telemedicine system according to claim 8, characterized in that the at least one communication module involves functions to record speech, pictures and video data and to transmit the data in real time.

10. A modular universal adapter telemedicine system according to claim 1, characterized in that the at least one function modules includes a locating module to locate the geographic position of the telemedicine system.

11. A modular universal adapter telemedicine system according to claim 10, characterized in that the locating module is a GPS module to determine and to transmit geographic position data.

12. A modular universal adapter telemedicine system according to claim 11, characterized in that the GPS module is integrated into the universal adapter.

13. A modular universal adapter telemedicine system according to claim 1, characterized in that the process module includes means to process, output and transmit data.

14. A modular universal adapter telemedicine system according to claim 1, characterized in that the at least one function module, and in and the universal adapter have their own internal battery.

15. A modular universal adapter telemedicine system according to claim 1, characterized in that the at least one function module and the universal adapter have a universal I/O connection through which at least one of the data transmission between the modules and charging of the function module's battery takes place.

16. A modular universal adapter telemedicine system according to claim 1, characterized in that the universal adapter includes a wireless interface, through which the data can be exchanged with at least one of the process module and the at least one function modules equipped with wireless interfaces, wherein the data transmission to the process module can just as easily be accomplished using a hard-wired I/O connection.

17. A modular universal adapter telemedicine system according to claim 1, characterized in that the universal adapter includes a function to automatically register connected function modules.

18. A modular universal adapter telemedicine system according to claim 1, characterized in that the at least one function module, and the universal adapter have a central processor and non-mechanical memory to store data at least temporarily.

19. A modular universal adapter telemedicine system according to claim 1, characterized in that the at least one function module has its own control elements and at least one item selected from the group consisting of an acoustic-/visual signal elements, function display and a display on the module.

20. A modular universal adapter telemedicine system according to claim 1, characterized in that the universal adapter has four control elements and at least one item selected from the group consisting of an acoustic-/visual signal elements, a function display and a display on the module.

21. A modular universal adapter telemedicine system according to claim 20, characterized in that the at least one function module, can be easily operated through two of the four control elements located on the universal adapter concerning basic functions.

22. A modular universal adapter telemedicine system according to claim 20, characterized in that the at least one function module and the universal adapter can be expanded in operation and configuration through the four control elements located on the universal adapter.

23. A modular universal adapter telemedicine system according to claim 1, characterized in that the universal adapter and the at least one function modules can be operated in different user modes.

24. A modular universal adapter telemedicine system according to claim 23, characterized in that user modes are available to at least one of the patient, the physician, multiple patients and remote access by the physician's receiving center.

25. A modular universal adapter telemedicine system according to claim 23, characterized in that the process module includes a function to change the user mode of the universal adapter.

26. A modular universal adapter telemedicine system according to claim 1 characterized in that the at least one function module can be used either [a] using the universal adapter and a process module to transmit data directly or [b] separately without the universal adapter.

27. A modular universal adapter telemedicine system according to claim 1, characterized in that the ability is available to perform wireless communication between the universal adapters of multiple modular universal adapter telemedicine systems, and to use a process module in common through a wireless or hard-wired transmission path to said process module.

28. A modular universal adapter telemedicine system according to claim 1, characterized in that diagnostic and therapeutic plan monitoring and medical monitoring software is integrated into the universal adapter.
